(19) United States
(12) Patent Application Publication
(10) Pub. No.: US 2008/0045914 A1
(43) Pub. Date: Feb. 21, 2008

(54) Diaper shredder, liquefier disposal

(76) Inventor: Zoltan Egeresi, Santa Cruz, CA (US)

Correspondence Address: ZOLTAN EGERESI, 5500 COAST RD., SANTA CRUZ, CA, 95060, US

(21) Appl. No.: 11/448,906

(22) Filed: Jun. 8, 2006

(51) Int. Cl. A61F 13/15

(52) U.S. Cl. 604/364

(57) ABSTRACT

The Liquid Window Shade is a sealed double glazed window comprises of two spaced panes of glass sealed in a frame with spacer with sealed holes for connecting hoses for dyed water or other colored liquid to be pumped in or re-circulated, to block light coming in or decrease it to increase privacy, UV filtering or decrease heat loss. Air pump is to create a stream of air curtain inside the enclosure using several holes in the air strip located on the bottom. Draining solenoid, liquid and air pump connected to a timer creates an automated “shade up, shade down” cycle. Different colors, waterproof confetti, UV filtering chemicals can be added, for seasonal changes, for parties, or creating a falling snow flake affect.

### CROSS REFERENCE RELATED APPLICATIONS

U.S. Pat. No. 3,952,745 by Duncan explains a flushable diaper, where the inner absorbent pad is enclosed with a different, stronger material which can not be flushed, requiring a messy separation. This type of self-dissolving pad could plug up the toilet if larger diaper is used or if the envelope cover is not removed.

### FEDERALLY SPONSORED RESEARCH AND DEVELOPMENT

Not applicable

### BACKGROUND OF THE INVENTION

### Field of the Invention

This invention relates to flushable diaper disposal, current US class 128/287, /284. It is well known, that disposable soiled diapers of various sizes are not flushable in the toilet due to the fact that the outer cover of the diaper is durable plastic, way to balky for any flushing attempt. In most cases soiled diapers are stored in garbage bag or can for several days before weekly garbage pick up occurs as in most places. Bacteria, viruses may be present when children are sick, odor lingers on especially during long hot days. My invention provides a solution for all size immediate diaper disposal, ability to shred, tear apart, pulverize the diaper in a diaper processor with running water emptying into the toilet to be flushed. This invention combines prior art shredders, food processors into a new art to deal with a long standing problem waiting for a solution.

### BRIEF SUMMERY OF THE INVENTION

The object of this invention is to create a novel way to dispose soiled disposable diapers. A water tight container with a saleable top attachable to toilet tank, connected to a controlled water source contains a shredder on the top to shred the diaper in the first stage, in the second stage vertically rotating claws tear the shredded pieces further apart to smaller pieces, in the third stage a horizontally rotating blade shreds and further pulverizing the diaper's inner and outer material and flushes away as the water is pumped into the diaper processor. Liquefied material is conducted into the toilet bowl ready to be flushed.

### BRIEF DESCRIPTION OF THE INVENTION

A sealable container (1) preferably attached to the side of toilet tank, large enough to handle the largest diaper has a sealable top(2) with a latch(27). Soiled diaper is placed inside of the diaper processor; shredder (20) cuts the diaper longitudinally as water is sprinkled onto the shredder (5) in the upper first stage. In the second stage the vertically rotating claws (19) tear apart the shredded diaper and pulls it down to the lower chamber (39). In the third stage a horizontally rotating blade (16) further shreds, pulverizes the material and spins it out at the out flow (14) into the toilet bowl.

### BRIEF DESCRIPTION OF SEVERAL VIEWS OF THE DRAWINGS

FIG. 1 of Sheet 1 of 2 shows the front view of the diaper shredder bidirectional shredder located on the upper section with water inlet, lower section illustrates the rotating blades.

FIG. 2 shows an ordinary toilet where the shredded diaper with water enters into with a connecting hose for flushing

FIG. 3 of sheet 2 of 2 shows side view of the upper section, the shredder with one wheel spring loaded to prevent jamming and spinning cutter.

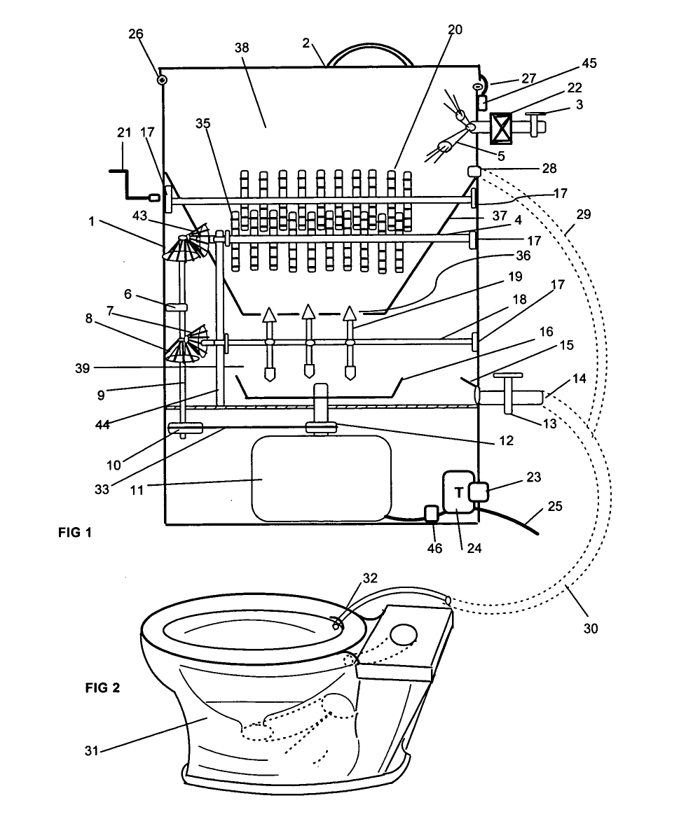

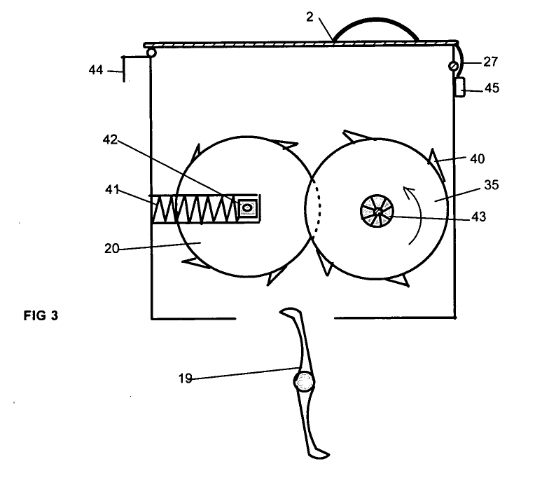

### DETAILED DESCRIPTION OF THE INVENTION

My invention comprises of a hermetically sealed double glazed window (14), two separated panes of glass sealed in with a frame (16) with spacer(13), at the bottom holes (9) are located to conduct liquid from the pump (3) to fill the space between the window panes. Liquid return flow is trough drain outlet (8) return line (31) via valve (33) and solenoid (7 to the liquid container (25) preferably located just in front of the Liquid Window Shade having enough capacity to hold the liquid required for the total in between window space volume. When the fluid container (25) is full, power coming from connector (1) trough timer (2) with one of the three controlled output turns on water pump using AC cable (24), colored/dyed fluid exits at the bottom of the liquid tank (25) at connection (32) via hose (5), adjusting valve (4), check valve (6) trough connection (9) pumping liquid into the hermetically sealed space (29). Timer (2) has at least three controlled outputs with manual override to be able to turn on and off liquid, air or solenoid as to set up liquid level, and special effects. Timer should be on till the window space is filled with liquid or until water level (15) reaches lever sensor (28) as it shuts the pump off, or liquid gets re-circulated trough overflow hole (30). When user wants to change color, or type of liquid, it can be drained off when drain switch (33) is opened and drain solenoid (7) is activated, open.

By using the timer to set up the liquid pumping time and set timing to open solenoid to drain the Window “pulling away the shade” the function of the automated Liquid Window Shade can be achieved as the length of shading time can be programmed. To set up the special effect of the invention the timer (or manual) controlled air pump (23) trough air hose (20), air volume valve control (22), check valve (21) air hole (19) air can be pumped in to sealed enclosure to create a bubble curtain as the bubbles (12) leave trough air holes (10), these bubbles can also steer up the water proof confetti (11) for additional shading or creating a snow fall effect. The liquid container (25) can be drained trough draining hole (26) and filled up at opening (27).

Liquid may also be replaced by theatrical fog, in this case the fog generator would replace the water and air pump re-circulating the fog making material.

In respect, after explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting. It is important, therefore, that the claims be regarded as including such equivalent constructions insofar as they do not depart from the spirit and scope of the present invention, nor is it intended to be limiting as to the scope of the invention in any way.

What I claim as my invention is Liquid Window Shade comprising of:

1. A hermetically sealed pair of glass enclosed in a frame with inner space capable to be filled with dyed or colored liquid, water, mineral oil or other liquid chemicals or theater fog providing capabilities to reduce heat loss and limit or block light, block or reduce UV light or sunlight to get trough to provide privacy;
a. The insulation between the panes having several hose connection for liquid to be pumped in, liquid be drained, overflow connection for liquid or theatrical fog re-circulation, air hose connection, liquid sensor for level control;
b. An air pump with a volume controller controlled by a timer or manually activated, capable of pumping sufficient amount of air into the bottom of the Liquid Window Shade to crate an air bubble stream, additional shading effect and steer up waterproof confetti, check valve providing protection against fluid entering into the air pump;
c. A shut off valve to prevent liquid back flow to the container if closed -manual operation-, with open valve the solenoid in the liquid return line is opened by the timer providing timed, automated draining, shade controlling;
d. A liquid container having sufficient capacity to hold enough liquid for the window's inner space, connection for drain return line, pump, overflow return line, for container filling.
